(19) United States
(12) Patent Application Publication
(10) Pub. No.: US 2012/0225015 A1
(43) Pub. Date: Sep. 6, 2012

(54) Method for predicting autism spectrum disorders by cannabinoid and cannabinoid receptor expression

(76) Inventors: Stephen T. Schultz, San Antonio, TX (US); Mauris N. DeSilva, Austin, TX (US); Georgianna G. Gould, San Antonio, TX (US)

(21) Appl. No.: 13/411,538

(22) Filed: Mar. 3, 2012

Related U.S. Application Data
(60) Provisional application No. 61/448,891, filed on Mar. 3, 2011.

(51) Int. Cl. A61K 51/04; G01N 27/62; A61K 49/00

(52) U.S. Cl. 424/1.65; 424/9.1; 436/501

(57) ABSTRACT

The inventive method relates to a method for the determination of susceptibility or diagnosis of autism or autism spectrum disorders. Diagnosis or determination of susceptibility determinations are predicated on quantitative analysis of endocannibinoid levels or endocannibinoid receptor expression.

### CROSS-REFERENCES TO RELATED APPLICATIONS

This application claims the benefit of U.S. Provisional Application No. 61/448,891, filed 3 Mar. 2011.

### BACKGROUND OF INVENTION

1. Field of Invention

The inventive subject matter relates to a method of diagnosing or predicting the propensity for autism using endogenous cannabinoids and/or cannibinoid receptor expression.

2. Background Art

Autism spectrum disorders (ASD) are a spectrum of psychological conditions characterized by social interaction and communication deficits. Symptoms also include repetitive behavior that appear early in childhood, usually before age 3 years and often are accompanied by abnormalities in cognitive functioning. CDC, MMWR, 58 (SS10:1-20 (2009)) and American Psychiatric Association, Diagnostic and Statistical Manual of Mental Disorders (4^{th} ed), Washington, D.C. (1994). The prevalence of autism in the United States is approximately 1 in every 110 births (1 in 70 boys). CDC, MMWR, 58 (SS10:1-20 (2009).

ASD encompass a range of behaviorally defined conditions. The five forms of ASD, including: Autism; Asperger syndrome; atypical autism (pervasive developmental disorder-not otherwise specified (PDD-NOS); Rett syndrome; and Childhood Disintegrative Disorder. Asperger syndrome is closest to autism in signs and causes. Rett syndrome and Child Disintegrative Disorder have similar symptoms as autism, but their etiology may be unrelated. Volkmar, et al., J. Child Psychol. Psychiatry, 50: 108-15 (2009).

Two of the prominent features of autism are immune system dysregulation (Pessah, et al., Neurotoxicology, 29:532-545 (2008) and abnormal brain neuron organization (Courchesne, et al., Neuron, 56:399-413 (2007)).

Autism can be co-morbid with tuberous sclerosis (1.2%), fragile X syndrome (0.3%), and congenital rubella syndrome (0.3%), although the attributable proportion of all medical disorders is less than 10%. However, in most cases, the cause of autism is unknown (Fombonne, e., J. Autism Dev. Disord., 33:365-382 (2003)).

### SUMMARY OF THE INVENTION

The current inventions relates to a method of determining indicia of autism or ASD by quantitating endocannibinoid levels.

An object of the invention is a method of determining the susceptibility of autism or ASD using biological markers comprising measuring endocannabinoid levels.

Another object of the invention is a method of diagnosing autism or ASD by quantitating endocannabinoid levels in a patient. The endocannibinoids comprise either one or more of Δ⁹-tetrahydrocannabinol (THC); N-arachidonoylethanolamine (anandamide); N-palmitoylethanolamine (PEA); cannabidiol (CBD); 2-arachidonoyl glycerol (2-AG); and N-oleoylethanolamine (OEA),

Another object is to measure endocannibinoids subsequent to acute exposure to acetaminophen or sociability testing.

A further object of the invention is the measurement of endocannibinoid levels, in a diagnostic method or method to determine susceptibility to autism or ASD by quantitating endocannabinoid receptors.

A still further object of the invention is the determination of susceptibility or diagnosis of autism or autism spectrum disorder by quantitating endocannabinoid synthesis and turnover.

### BRIEF DESCRIPTION OF DRAWINGS

FIG. 1. Acetaminophen was administered by intraperitoneal injection 50 minutes prior to testing in the three-chambered social approach task. Bars show the mean+/−S.E.M. of each treatment group for this and all subsequent graphs. N=6 mice per dose. Acetaminophen doses of 100 mg/kg and 400 mg/kg increased dwelling near confined stranger mice by adult male BTBR.

FIG. 2. Effect of acetaminophen on social interaction marble burying behavior in BTBR mice. (a) Acetaminophen (ACM, 100 mg/kg) increased dwelling near stranger mice relative to vehicle-control (CTRL, saline±10% DMSO) treated BTBR mice (* p<0.05), while WIN 55, 212-2 (WIN) treatment increased lingering in the arena center (** p<0.05). (b) There was no difference among drug treatment groups in time engaged in social sniff of stranger mice (black bars) or time investigating the empty cup cage (white bars) during social interaction approach testing. (c) Administration of acetaminophen and WIN 55, 212-2 reduced time spent in the chamber of the arena with a novel stranger (* p<0.05), and in this social novelty test WIN 55, 212-2 reduced dwelling in the arena center (** p<0.05). (d) Marble burying behavior was similar among BTBR mice, irrespective of drug treatment. N=7-14 mice per treatment group.

FIG. 3. Effect of acetaminophen on social novelty and marble burying behavior in 129S1/SvImJ mice. (a) Drug-treated and control (CTRL) mice spent similar amounts of time by stranger mice in a cage vs. an empty cage (novel object) in the social approach test. However WIN 55, 212-2 (WIN) treatment increased time spent by 129S mice in the arena center (**p<0.05), and reduced their exploratory behavior, as indicated by fewer chamber entries. (b) There was no difference in time spent sniffing stranger mice or empty cages among drug treatment groups during the social approach test. (c) Acetaminophen (ACM) increased dwelling near new strangers relative to controls, while WIN 55, 212-2 increased dwelling in the center chamber during the social novelty test (p
<0.05 for each). (d) Acetaminophen treatment increased marble burying behavior relative to control or WIN 55,212 treatments (*p<0.05). N=9 mice per group.

FIG. 4. Anandamide levels in the frontal cortex of (a) BTBR mice or (b) 129S1/SvImJ mice 70 minutes following injection, or injection plus sociability testing. In (a) and (b), an (*) indicates significantly different from saline-injected controls that were not subjects in sociability testing.

FIG. 5. Effect of daily injections (×59 days) of early development (18-21 days postnatal) BTBR and C57BL/6 mice of acetaminophen, valproic acid or WIN 55, 212-2 on marble burying behavior.

FIG. 6. 2-AG levels after daily treatment of early postnatal development mice with acetaminophen, valproic acid or WIN 55,212-2

### DETAILED DESCRIPTION OF PREFERRED EMBODIMENTS

The following terms are defined:

“Autism Spectrum Disorder” refers to a group of developmental disabilities that includes: Autism; Asperger syndrome; pervasive developmental disorder not otherwise specified (PDD-NOS or atypical autism); Rett syndrome; and childhood disintegrative disorder.

“Autism” refers to an autism spectrum disorder characterized by a neural development disorder leading to impaired social interaction and communication by restricted and repetitive behavior.

“Cannabinoid” refers to a class of chemical compounds including phytocannabinoids, which are oxygen-containing C₂₁ hydrocarbons found in the plant species Cannabis sativa (i.e., marijuana), including metabolites and synthetic analogues thereof. Cannabinoids include, but are not limited to, cannabinol, Δ⁹—tetrahydrocannabinol (THC) and cannabidiol). Cannabinoids as used here also refer to chemical compounds which mimic the actions of phytocannabinoids or have similar structure, such as endocannabinoids. Cannabinoids include compounds that have a high affinity for the cannabinoid receptor, such as THC and those cannabinoids that do not, such as cannabidiol.

Endocannabinoids refer to a class of compounds with similar structure to phytocannabinoids but that are found in animals and that activate cannabinoid receptors.

“Diagnostic” refers to identifying the nature of a pathologic condition.

“Susceptibility of autism or autism spectrum disorder” refers to the likelihood of currently exhibiting or exhibiting in the future autism or one of the other autism spectrum disorders.

Endocannabinoids serve as intercellular, neuromodulatory lipid signals. They are involved in a variety of physiological processes including appetite, pain-sensation, mood and memory. Endocannabinoids are derivatives of arachidonic acid, as well as other poly-unsaturated fatty acids.

Endocannabinoids are endogenous ligands for cannabinoid receptors that are also bound by exogenously introduced cannibinoids, such as THC, the psychoactive component of Cannabis sativa (i.e., marijuana). The cannabis receptors are classified into two groups, CB₁ and CB₂.

Endocannabinoids include anandamide (N-arachidonoylethanolamine (AEA)), Δ⁹-tetrahydrocannabinol (THC), N-palmitoylethanolamine (PEA), cannabidiol, N-oleoylethanolamine (OEA), and 2-arachidonoylglyceraol (2-AG). AEA is produced mainly by a transacylase-phosphodiesterase-mediated pathway, initiating from the precursor N-arachidonoyl-phosphatidylethanolamine. The biosynthesis of 2-AG proceeds by rapid hydrolysis of inositol phospholipids by specific phospholipase C generating diacylglyceraol (DAG), which is converted to 2-AG by a sn-1-DAG lipase.

AEA and 2-AG act primarily via cannabinoid receptors. There are seven trans-membrane spanning receptors that belong to the rhodopsin family of G protein-coupled receptors. The binding of endocannabinoids to CB receptors triggers various signaling pathways, such as the inhibition of adenylyl cyclase, the regulation of ionic currents, the activation of focal adhesion kinase, of mitogen-activated protein kinase (MAPK) and of cytosolic phospholipase A2 and the activation of (CB1) or the inhibition (CB2) of nitric oxide synthetase. Both AEA and 2-AG are presumably taken up by cells through a specific carrier, which has not yet been clearly identified. Once inside cells, the cannabinoids are metabolized by multiple pathways.

Plasma endocannabinoid levels have been determined in humans and correlated with a number of maladies, including circulatory disfunction (Quercioli, et al., European Heart Journal 32: 1369-1378 (2011)), and mental health disorders such as anorexia nervosa (Monteleone, et al., Neuropsychopharmacology 30: 1216-1221 (2005)).

The endocannabinoid system plays an important role in the development of the central nervous system. Activation of the endocannabinoid system can induce long-lasting physiological responses (Campolongo et al. Int. Rev. Neurobiol., 85:117-133 (2009)). Use of cannabis (i.e., exogenous cannabinoid) in the still-maturing brain may produce persistent alterations in brain structure and cognition (Jager and Ramsey, Curr. Drug. Abuse Rev., 1: 114-123 (2008)). Animal models have revealed the danger of both cannabis abuse and exposure to cannabinoid drugs during brain development (Anavi-Goffer and Mulder, Chembiochem., 10:1591-1598 (2009)).

Cannibinoid receptors can be classified into two know groups, CB₁ or CB₂. Developmental problems associated with the endocannabinoid system may occur through either of these two receptor classes.

CB/receptors are located in the central nervous system (CNS), peripheral nervous system, and peripheral organs. In the CNS, CB₁ receptors are concentrated in the cerebellum, hippocampus, and the basal ganglia (Drysdale and Platt, Curr. Med. Chem. 10:2719-2732 (2003)), which are areas in the brain implicated as important in autism (Courchesne et al., Neuron 56:399-413 (2007), Bauman and Kemper, Int. J. Dev. Neurosci. 23:183-187 (2005)). During fetal development, CB₁ receptors activation is important for neuron differentiation and proper axonal migration (Pride et al., Vitam. Horm. 81:139-158 (2009)). In addition, recent studies suggest that CB₁ receptors define synapse positioning (Harkany et al., Cur. Opin. Neurobiol. 18:338-345 (2008)). Modulation of CB₁ cannabinoid receptors may also trigger autism by interrupting normal brain development.

CB₂ receptors are primarily located in immune tissues and cells and may serve a regulatory function. CB₂ receptors have been implicated with regulation of movement of inflammatory cells to the site of injury (Lunn et al., Br. J. Pharmacol. 153:226-239 (2008)). The activation of CB₂ receptors may also slow the progression of Alzheimer's disease by stimulating beta-amyloid removal by macrophages (Tolon et al., Brain Res. 1283:148-154 (2009)).

In a preferred embodiment, diagnosis of autism spectrum disorder or autism is determined by a method comprising quantitating endocannibinoid levels. In this embodiment, endocannibinoid levels are quantitated from serum samples collected from patients, ensuring against dietary fluctuations in endocannibinoids (Monteleone, et al., Neuropsychopharmacology, 30: 1216-1221 (2005); Habayeb, et al., JAMA 299: 1135-1136 (2012); Zoerner, et al., Anal Bioanal Chem. (online version: doi:10.1007/s00216-5729-9) (published 4 Feb. 2012)). Deviations of endocannibinoid levels, compared to normal levels, is indicative of autism spectrum disorder or susceptibility to the disorder.

In another embodiment, endocannibinoid levels can be indirectly assessed by quantitating cannibinoid receptor levels. The embodiment avoids any variability inherent in serum endocannabinoid levels. Quantitation of receptors has the added potential advantage of improved accuracy since low levels of endocannibinoids can induce (i.e., upregulate) endocannibinoid receptors in the brain.

In a preferred embodiment, a diagnosis or determination of susceptibility to autism or autism spectrum disorders is made in individuals by first administering high doses of acetaminophen (Kozer, et al., Acta Paediatr., 95: 1165-71 (2006)), typically 12 mg/kg. Alternatively, individuals are subject to sociability testing. Afterwards, the level of plasma endocannibinoids is quantitated. Endocannibinoids include Δ⁹—tetrahydrocannabinol (THC); N-arachidonoylethanolamine (anandamide); N-palmitoylethanolamine (PEA); cannabidiol (CBD); 2-arachidonoyl glycerol (2-AG); and N-oleoylethanolamine (OEA), wherein said diagnosis or determination of susceptibility is made if the quantitated endocannibinoid level of said individual is different from the mean of the range of individuals, of the same age group, without autism or autism spectrum disorders.

In another embodiment, a diagnosis or determination of susceptibility to autism or autism spectrum disorders is made if the 2-AG level is at least twenty percent (20%), or for AEA, if the AEA level is forty five to seventy five percent (45%-75%), above or below the mean level for non-autistic individuals or individuals without autism spectrum disorders.

### Example 1

### Anandamide Levels as Indicator of Autistic Behavior

Autism is associated with impairments in social interaction; communication; and restricted interests and repetitive behavior. The ability to study the physiological and molecular mechanisms associated with this disease is hampered by suitable models. However, several inbred mouse strains demonstrate inherent behaviors paralleling these sociability impairments. These include the strains BTBR and 129S1/SvImJ (Moy, et al. Behav. Brain Res., 176: 4-20 (2007); McFarlane, et al., Genes Brain Behav., 7: 152-163 (2008); Defensor, et al., Behav. Brain Res., 217: 302-308 (2010); Spencer, et al., Autism Res., 4: 40-56 (2011)).

The social behavior of BTBR mice is sensitive to changes in serotonin (5-HT) neurotransmission and anxiolytics (Gould, et al., J. Neurochem., 116: 291-303 (2011)). Furthermore, administration of the endocannabinoid agonist anandamide or high doses of the pain reliver acetaminophen promote social interactions in mice (Umathe, et al., Prog. Neuropsychopharmacol Biol. Psychiatry, 33: 1191-1199 (2009). Although acetaminophen has little to no affinity for 5-HT receptors, its administration raises 5-HT levels in brain tissue and downregulates 5-HT₂ receptors (Courade, et al., Naunyn Schmiedebergs Arch. Pharmacol., 364: 534-537 ((2001); Sandrini, et al., Inflamm. Res., 56: 139-142 (2007)).

CB₁ receptors are bound on cell bodies and axons of 5-HT neurons and CB agonists such as WIN 55, 212-2 inhibit presynaptic 5-HT release in brain (Nakazi, et al., Naunyn Schmiedebergs Arch. Pharmacol., 361: 19-24 (2000); Lau and Schloss, Eur. J. Pharmacol., 578: 137-141 (2008)). Acetaminophen's effects on social behavior and analgesic activity are likely to be mediated through its metabolic deacetylation products para-aminophenol and/or N-arachidonoylphenolamine (AM404). These metabolites likely activate CB receptors directly, or indirectly by raising extracellular endogenous CB levels in the brain (Högestätt, et al., J. Biol. Chem., 280: 31405-31412 (2005); Ottani, et al., Eur. J. Pharmacol., 531: 280-281 (2006); Bertolini, et al., CNS Drug Rev., 12: 250-275 (2006); Mallet, et al., PLoS One., 5: e12748 (2010)).

Studies were conducted to determine the affects of acute administration of acetaminophen on social interaction and endogenous cannabinoid levels in the anterior cingulated region of the frontal cortex. These studies were conducted using the socially-impaired mouse strains BTBR and 129S1/SvImJ. The cingulated cortex was targeted because serotonergic tone in this region is linked to anxiety and emotional states that shape social behavior.

BTBR T+tf/J, 129S1/SvImJ and C57BL/6 mouse colony founders were originally obtained from the Jackson Laboratory (Bar Harbor, Me., USA). These strains were bred in the animal facilities of the University of Texas Health Science Center at San Antonio through 2 generations. After weaning at 23-25 days of age, male littermates were housed in groups of 4-5 per cage until behavioral testing at 3-4 months of age. Mice had ad libitum access to food (Teklad™ rodent diet, Harlan, Indianapolis, Ind., USA) and water in ventilated clear plastic cages lined with chipped wood bedding. The housing room had a 12 h light/dark cycle (lights on/off at 7:00) and was maintained at 20-22° C.

Mice were administered acetaminophen (1-400 mg/kg, Sigma, St. Louis, Mo., USA) or 0.9% saline solution by intraperitoneal (i.p.) injection. The cannabinoid agonist WIN55, 212-2 (Ascent Scientific, Princeton, N.J., USA) was dissolved initially in dimethyl-sulfoxide (DMSO Sigma) and was diluted with saline (1:10) to administer 0.1 mg/kg i.p. in 10% DMSO to mice. A sub-group of control mice were treated with 10% DMSO in saline vehicle, these mice did not differ significantly from saline-treated mice in behavioral tests (F_{1.7}<1.25; p>0.3 for all parameters), so the two treatment groups were pooled. Injections were given 30 minutes prior to introduction to the testing arena for conditioning, and 50 min prior to behavior testing.

The three-chamber sociability testing procedure for mice is described in detail in Yang et al. Curr. Protoc. Neurosci., Chapter 8: Unit 8. 26 (2011), our study was conducted in a manner consistent with that protocol. Mice were introduced into the center chamber of an acrylic three-chamber sociability arena, measuring 30×22×61 cm with a light tan bottom, black side walls and two transparent interior walls with slat door openings of 10 cm² spaced 17 apart dividing the arena into 3 chambers, for 20 min prior to behavioral testing. As described in Gould et al. J. Neurochem., 116: 291-303 (2011), pre-conditioning was performed under low red light (16 lux) at first for 10 min with the mouse confined in the center chamber, then with the doors opened so the subject could explore the entire arena for 10 min longer. Just prior to testing, subjects were briefly confined in the center chamber while an empty wire cup cage was placed at one end of the arena, and a stranger mouse of the same strain was placed under an identical cup cage at the opposite end. Stranger mice were neither litter- nor cage-mates of the subjects and were housed in a separate ventilated cage rack. Stranger mice were pre-conditioned under cup cages in 3 sessions of 30 min each in the day(s) prior to testing, separated by 1 hour reprises in their home cages. Cup cages were topped with weighted jars (9 cm high×7 cm diameter) to prevent mice from climbing on top of them. Digital video cameras (Hewlett-Packard Photosmart™ R742, Palo Alto, Calif., USA) positioned on top of tripods overseeing the arenas were turned on, the doors were removed and social approach behavior was recorded for 10 min under low red light.

The social approach test ended with confinement of the subject into the center chamber and closing of the slat doors. A new stranger mouse was then placed under the empty cup cage, the doors were opened, and behavior was recorded for another 10 min under low red light to assess preference for social novelty. At the end of the test, stranger mice were returned to their home cages for use in subsequent tests, and subject mice were removed from the arena and placed in a marble burying test. The number of boli in the center of the arena was counted prior to cleaning the arena with a solution of 70% EtOH and paper towels prior to conditioning and testing the next animal. Digital videos were analyzed for box entries, time in box and social sniffing time by observers blind to mouse strain or drug treatments.

Marble burying was assessed in a dark room (<16 lux) by placing 15 or 20 blue marbles on top of fresh wood chip bedding filled to a depth of 4-5 cm in a 22×42 cm clear acrylic rat cage covered with a filter top. Mice were placed in the cages to bury marbles for 30 min. Marbles that were at least ⅔ covered by bedding were considered buried, as described previously (Gould et al., J. Neurochem., 116: 291-303 (2011)). Following the marble burying task, mice were sacrificed by cervical dislocation and decapitation; their brains were removed and frozen on powdered dry ice. Cingulate cortex was isolated and stored at −80° C. for subsequent measurement of endocannabinoid levels.

To measure levels of the fatty acid amides 2 arachidonyl glycerol (2-AG), anandamide (AEA), and oleoylethanolamide (OEA), frozen cingulate cortex samples were spiked with 50 pmol of [²H₄]anandamide, [²H₄]oleoylethanolamine and [²H₅]-2-arachidonyl glycerol (internal standards) and processed as in Hardison et al., Prostaglandins Other Lipd Mediat., 81: 106-112 (2006). Briefly, lipids were extracted by adding methanol/chloroform/water (1:2:1, v/v/v) and the chloroform layer was further purified by solid phase extraction using C18 Bond Elut cartridges (100 mg; Varian, USA). Endocannabinoid-containing fractions were analysed by gas chromatography/chemical ionization mass spectrometry (GC/MS) using an isotope dilution assay as described in Seillier et al. Int. J. Neuropsychopharmacol., 3: 373-386 (2010).

### Effects of Acetaminophen in Socially-Deficient Mice

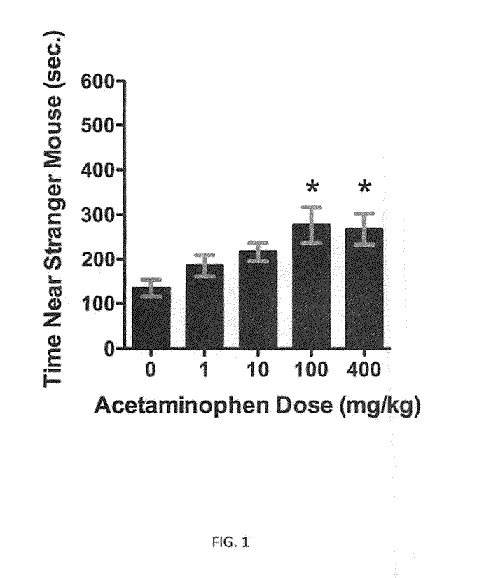

The dose-response relationship for acetaminophen (1-400 mg/kg i.p.) to promote dwelling near a stranger mouse in the three chamber social approach test was initially determined in adult male BTBR mice. The lowest dose of acetaminophen to significantly increase time spent in the chamber with a stranger mouse above that of saline-injected controls was 100 mg/kg (F_{4.25}=4.6, p<0.01), as illustrated in FIG. 1.

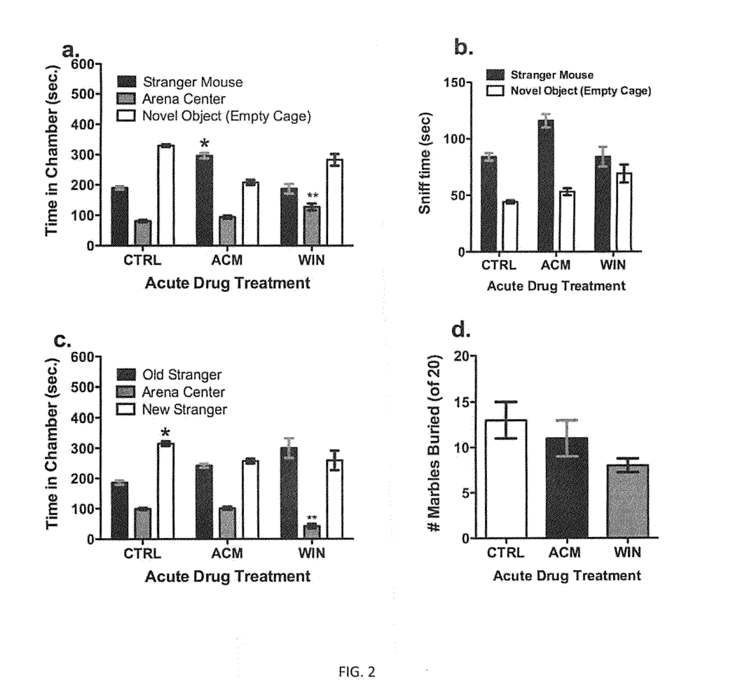

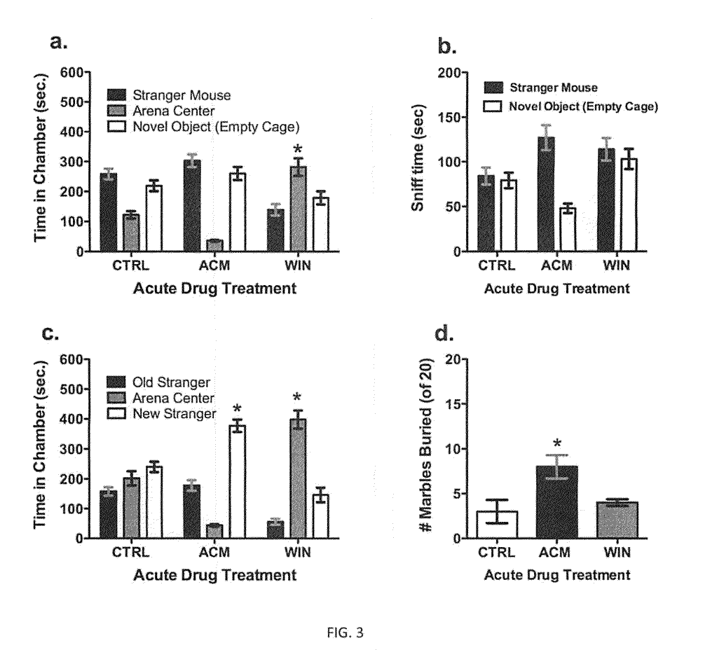

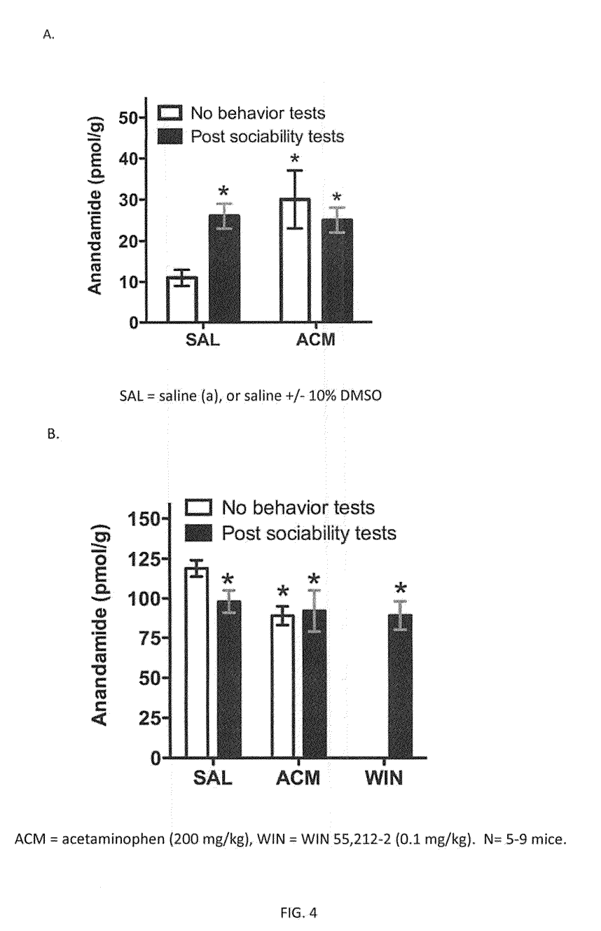

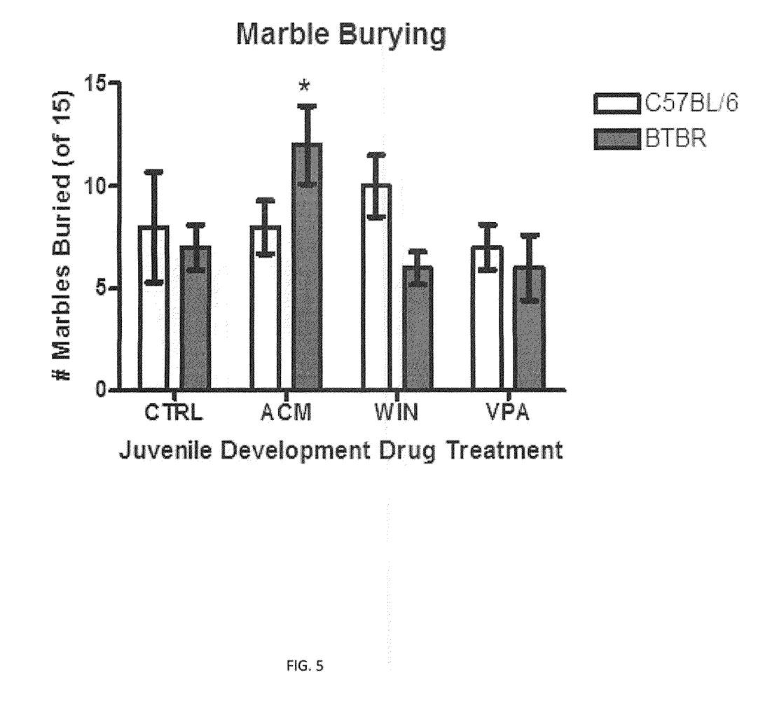

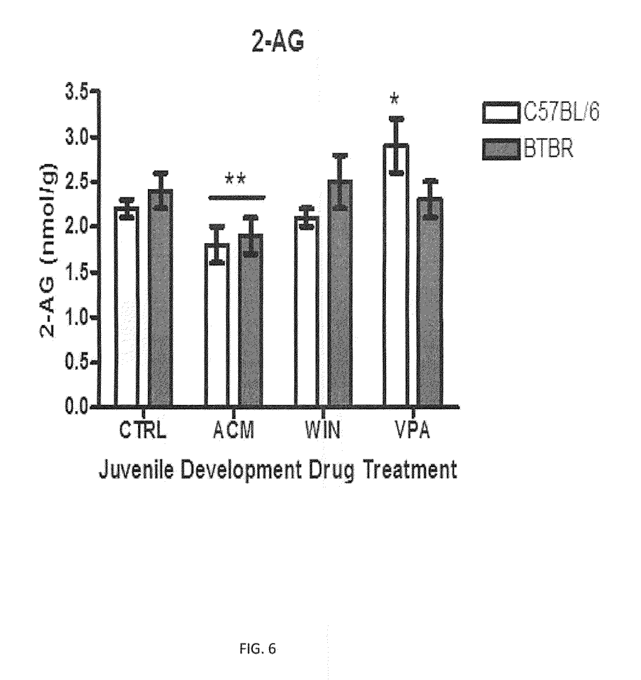

In subsequent three-chamber sociability tests, global repeated-measured ANOVA revealed significant interactions among acute drug treatments, test phase (social approach vs. social novelty) and duration of time spent in each side chamber (F_{INTERACTION 2.31}=7.21, p<0.005). In the social approach test, there was a significant interaction between drug treatment and chamber preference in adult male BTBR mice as determined by mixed-model ANOVA (F_{INTERACTION 2.31}=5.3, p<0.01), Acetaminophen (100 mg/kg) treated BTBR mice spent more time in the chamber with the stranger mouse and less time in the chamber with the empty cage than either vehicle controls (saline±10% DMSO) or WIN 55, 212-2 (0.1 mg/kg) treated mice (F_{2.31}≧4.8. p<0.025) (FIG. 2a). However, there were no differences among drug treatment groups in time engaged in social sniff of the stranger mouse, or investigation of the empty cage, during the social approach test (F_{2.31}<1.1, p=0.35 for both comparisons, see FIG. 2b). Chamber entries did not differ among drug treated BTBR mice, and were on average 41±5 during the 10 min social approach test. In the social novelty test, mixed model ANOVA revealed a significant interaction between drug treatment and chamber preference (F_{INTERACTION 2.31}=3.4, p<0.05). Both acetaminophen and WIN 55.212-2 treated mice spent less time in the box with the novel stranger than controls (F_{1.31}=40, p<0.001), and WIN 55, 212-2 treated mice spent less time than the other groups in the arena center (F_{2.31}=3.4, p<0.05), as shown in FIG. 2c. Chamber entries for WIN 55,212-2 treated BTBR mice were lower (19±3) than either vehicle-control (45±5) or acetaminophen-treated (50±7) mice (F_{2.31}=5.4. p<0.01) during the social novelty test. Although there was a trend toward WIN 55212 treated mice burying fewer than controls, there was no significant difference in marbles buried by BTBR mice among treatment groups (F_{2.17}=2.4. p=0.12. FIG. 2d).

Adult male 129S1/SvImJ (129S) mice exhibited global differences in chamber dwelling patterns among drug treatments (F_{2.24}=7.4, p<0.003) and across the two sociability test phases (F_{1.24}=5.0, p<0.03), without interaction, in repeated-measures ANOVA comparisons. In the social approach test, all groups spent essentially equal time in the chamber with a stranger mouse and in the chamber with an empty cage (FIG. 3a). However, 129S mice treated acutely with WIN 55,212-2 (0.1 mg/kg) spent more time than acetaminophen (100 mg/kg) or vehicle (saline±10% DMSO) treated mice in the center chamber (F_{2.24}=5.0, p<0.02). WIN 55, 212-2 treated mice also made fewer chamber entries on average (9±4), than either acetaminophen treated (21±5) or control mice (27±5) during the social approach test (F_{2.24}=3.6, p<0.05). Time spent sniffing the stranger mouse or investigating the empty cage did not differ among drug treatment groups (FIG. 3b.), although there was a trend toward acetaminophen-treated 129S mice spending more time investigating strangers that did not reach significance (F_{2.24}=2.5, p=0.1). In the social novelty phase (FIG. 3c.), acetaminophen-treated 129S mice spent significantly more time than controls in the arena chamber with a new stranger mouse, while WIN 55,212-2 treatment increased dwelling in the center chamber of the arena (F_{2.24}>3.0, p<0.05). The number of chamber entries in the social novelty test was similar across drug treatment groups, and averaged 21±4 for all 129S mice over 10 min. Marble-burying in acetaminophen-treated mice was greater than in vehicle-control or WIN 55,212-2 treated 129S mice (F_{2.24}=4.0, p<0.05), as shown in FIG. 3d.

BTBR mice that were either treated with acetaminophen and returned to home cages, or were saline-treated subjects in sociability tests had significantly higher anandamide (AEA) levels in frontal cortex (drug effect and interaction F_{1.19}>4.11, p≦0.05, Fisher's LSD post hoc p<0.05, N=5-7) than saline-treated controls, but these effects were not additive, as shown in FIG. 4a. Oleoylethanolamide (OEA) levels did not differ among treatment groups (effects and interaction F_{1.8}<2, p>0.18), and were on average 65±6 pmol/g. Levels of 2 arachidonyl glycerol (2-AG), also did not differ significantly among treatments, in these studies. However, there was a trend (behavior effect F_{1.20}=3.66, p=0.07) toward the BTBR mice that performed sociability tasks having slightly higher 2-AG levels (4.7±0.3 nmol/g) than those that did not (3.9±0.2 nmol/g).

In contrast, neither sociability testing nor acetaminophen treatment increased anandamide levels in the frontal cortex of 129S1/SvImJ (129S) mice. Instead, all treatments reduced anandamide levels by ˜20% relative to saline treated, behavior naïve controls (drug effects F_{1.25}=4.29, p<0.05; Fisher's LSD p<0.05), as shown in FIG. 4b. OEA levels were similar among 129S treatment groups, and were 82±4.5 pmol/g on average (F_{1.25}<1.0, p=0.33). However, 2-AG levels were higher (F_{1.25}=15, p<0.001, Fisher's LSD p<0.001) in saline-treated sociability test subjects (3.5±0.2 nmol/g) than in all other 129S groups (2.3±0.2 nmol/g).

In other studies, in BTBR mice, acetaminophen had significant affect on 2-AG levels. In these studies, BTBR and C57BL/6 mice, in early (18 to 21 days postnatal) development, were administered either 4 daily saline or 10% DMS/saline injections; 4 daily injections (i.p.) of 100 mg/kg acetaminophen; 1 injection (i.p.) of 400 mg/kg valproic acid or 4 daily injections (i.p.) 0.01 mg/kg of WIN 55,212-2 for 2 months (i.e., 59 days). The mice were then tested for marble burying and evaluated for neurochemical measures. As shown in FIG. 5, acetaminophen induced significant marble burying behavior. Coincident to this behavior was a diminution of 2-AG levels, as shown in FIG. 6.

The results show that acute changes in social behavior of adult male mice, with inherently low sociability, are modulated by indirect activation of C_{B1} receptors by elevated levels of the endocannabinoids anandamide in BTBR mice and 2-AG in 129SvImJ mice in the cingulate region of the frontal cortex. As such, the level of anandamide is an indicator of social disfunction associated with autism spectrum disorders.

The pain-relieving properties of acetaminophen appear to be mediated, in part, through cannabinoid CB₁ receptor activation and serotonin (5-HT) system modulation. Acetaminophen is unlikely to act as a direct agonist at CB₁ receptors, instead the FAAH inhibitor/CB ligand AM404 is produced through its metabolism, and AM404 increases levels of endogenous cannabinoids such as anandamide and 2-AG in extracellular fluid to activate CB₁ receptors indirectly (Högestätt et al., 2005; Bertolini et al., 2006; Schultz, 2010). Based on the results of these studies, quantitation of endocannibinoids, such as annadamide, are of value in diagnosing or determining susceptibility to autism spectrum disorders.

### Example 2

### Endocannabinoid Receptor Level Analysis

Cannabinoid receptors modulate serotonin signaling in the cingulated cortex region. Serotonergic tone is greater in the frontal cortex of fatty acid amide hydrolase (FAAH) knock-out mice. Their social behavior is enhance, presumably due to higher levels of endogeneous cannabinoid-agonist anandamide (Cassano, et al., Pschopharmacology. 214: 465-476 (2011)). In CB1 knock-out mice serotonergic tone is low, extracellular serotonin levels and serotonin synthesis are elevated in the frontal cortex with a reduced sociability in stressful new habitats relative to wild-type mice (Aso, et al., J. Neurochem. 109: 935-944 (2009); Haller, et al., Eur. J. Neurosci., 19: 1906-1912 (2004)).

Endocannibinoids can be indirectly assessed by measuring expression of endocannibinoid receptors. In a preferred embodiment, positron emission tomography (PET) is used to measure CB₁ receptor levels in the brain. In other embodiments, PET can be combined with computed tomography (CT) or magnetic resonance imaging (MRI) scans. This embodiment will enable discernment of receptor expression as a function of anatomical structure.

PET is a nuclear imaging technique that produces three dimensional images of functional processes in the body. Typically, the system is used for the detection of gamma rays emitted indirectly by a position-emitting radionuclide tracer, which is introduced into the body attached to a biologically active molecule.

An embodiment of the inventive method is the analysis of cannibinoid receptors following the introduction of radio-labeled endocannibinoid receptor ligand, such as anandamide or 2-AG, or functional analogs of these molecules, into patients. Although any radio-labeled tracer, suitable for PET can be used, in a preferred embodiment, radio-labeled agonists of CB₁ receptors, or their analogs, can be utilized, such as taranabant (N-[(1S,2S)-3-(4-Chlorophenyl)-2-(3-cyanophenyl)-1-methylpropyl]-2-methyl-2-((5-(trifluoromethyl) pyridine-2-yl)oxy)propanamide). As examples, the following are potential tracer molecules: In other embodiments, suitable radiolabels include: [¹¹C]CB-119; [¹¹C]JHU75528; [¹⁸F]MK9470; and [¹¹C]MePPEP (Hamill, et al., Mol. Imagining biol. 11:246-252 (2009)).

The raw data collected through PET (i.e., coincidence events) are then grouped into projection images (i.e., sonograms). After raw data collection, the data is pre-processed in order to remove artifacts, such as: random coincidences, estimation and subtraction of scattered photons, detector dead-time correction and detector-sensitivity correction, etc. Reconstruction of the final image is conducted by any number of available algorithms. These include filtered back projection and iterative expectation-maximization algorithms. In a preferred embodiment, correction for differential attenuation of photons will be corrected.

Receptors levels determined through analysis of PET imaging will be directly compared to that for normal, non-autistic levels. Higher cannabinoid receptor levels, compared to receptors levels found in children of similar age, is diagnostic of autism or ASD or susceptibility of disease.

### Example 3

### Diagnosis of Autism or ASD by Endocannabinoid Metabolite Turnover

Studies suggest that serotonin is important for aspects of prenatal and postnatal brain development (Chugani, D. D., Mol. Psychiatry. 7:S16-S17 (2002); Gasper, et al, Nat. Rev. Neurosci 4:1002-1012 (2003)). Serotonergic function has been reported in children with autism (Hoshino, et al., Neuropsychobiology 11:22-27 (1984); Anderson, et al., J. Child Psychol. Psychiatry 28:885-900 (1987); Cook, et al., J. Neuropsychicatry Clin. Neurosci. 2:268-274 (1990). It was subsequently shown that serotonin synthesis is altered in patients with autism (Chandana, et al., Int. J. Devl. Neuroscience 23:171-182 (2005). In these studies, children with autism exhibit a difference in the change with age in whole brain serotonin synthesis capacity, compared to age matched non-autistic children. Additionally, autistic children also exhibited abnormal cortical asymmetries of serotonin synthesis affecting either the left or right cortex.

The current method utilizes, as indicia of autism or ASD, direct quantitative analysis of endocannibinoid levels. In one embodiment, serum samples of patients are collected and the endocannibinoid levels quantitated by a number of techniques. Examples of quantitation methods include, but are not limited to, liquid chromatographic and mass spectroscopy (Palandra, et al, 0.1. Chromatog B. 887:2052-2060 (7009); Sipe, et PLoS ONE 5:e8792 (2010).

In an additional embodiment, quantitative assessment of endocannibinoids, is conducted by analysis of turnover of endocannibinoid metabolites. In this embodiment, radio-labeled precursors include any precursor of endocannabinoids. Examples include, but are not limited to, N-arachidonoyl phosphatidylethanolamine (NAPE); arachidonic acid and cephalin (AEA) and/or using the 2-AG precursor diacylglyceraol. Analysis of the conversion of these precursors can be quantitatively assessed by any of a number of ways. In one embodiment, assessment is conducted quantitation of radio-labeled endocannibinoids, bound to receptors, by PET. Alternatively, blood samples can be obtained and quantitative assessment of radio-labeled cannibinoids monitored by liquid chromatography and mass spectroscopy.

Alternative to quantitative assessment of metabolites, metabolic byproducts can be analyzed. For example, anandamide, an endocannibinoid, is hydrolyzed by fatty acid amide hydrolase (FAAH) into free arachidonic acid and ethanolamine. Arachidonic acid can be combined with p-aminophenol, a breakdown product of acetaminophen, to form AM404. In one embodiment, paracetamol can be administered to patients. Serum samples can then be analyzed for quantitative assessment of AM404 levels. Alternatively, radio-labeled paracetamol can administered and AM404 levels analyzed by PET.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. A method of diagnosing or determining susceptibility to developing, autism or autism spectrum disorder comprising administering acetaminophen or applying sociability testing to an individual and quantitating the levels of one or more endocannibinoids in said individual and comparing said level or levels with that of a normal individual, wherein said endocannibinoid is selected from the group consisting of Δ⁹-tetrahydrocannabinol (THC); N-arachidonoylethanolamine (anandamide); N-palmitoylethanolamine (PEA); cannabidiol (CBD); 2-arachidonoyl glycerol (2-AG); and N-oleoylethanolamine (OEA), wherein said diagnosis or determination of susceptibility is made if the quantitated endocannibinoid level of said individual is different from the mean of the range of individuals, of the same age group, without autism or autism spectrum disorders.

2. The method of claim 1, wherein said method further comprises administering acetaminophen prior to said quantitating endocannibinoid level and wherein a diagnosis or determination of susceptibility is made if the said N-arachidonoylethanolamine (anandamide) concentration plasma is changed at least forty five to seventy five percent (45% to 75%) or if the said 2-arachidonoyl glycerol (2-AG) level is changed at least twenty percent (20%) from the mean in non-autistic individuals or individuals without autism spectrum disorders of a similar age group.

3. The method of claim 1, wherein said quantitation of endocannibinoid level is by quantitating cannibinoid receptor levels

4. The method of claim 1, wherein said endocannibinoid levels are directly quantitated from patient sera.

5. The method of claim 1, wherein said determining of endocannibinoid levels by quantitating the level of AM404 synthesis.

6. The method of claim 1, wherein said endocannibinoid level is quantitated by analysis of endocannabinoid synthesis and turnover comprising the steps:
a. Administering radio-labeled endocannibinoid precursors into an individual;
b. Quantitating radio-labeled endocannibinoids

7. The method of claim 3, wherein said cannibinoid receptor levels are quantitated by a method comprising the steps:
a. Administering radio-labeled endocannibinoid, endocannibinoid analog or endocannibinoid precursor into an individual;
b. Detecting binding of said radio-labeled cannibinoid;
c. Comparing said binding of the radio-labeled cannibinoid to binding levels of non-ASD or autistic individuals.

8. The method of claim 5, wherein quantitating of AM404 comprises the additional steps comprising:
a. Administering radio-labeled AM404 precursor to a individual;
b. Collecting serum samples from said individual;
c. Quantitating radio-labeled AM404 by liquid chromotography.

9. The method of claim of claim 5, wherein quantitating of AM404 comprises the additional steps comprising:
a. Administering radio-labeled AM404 precursor to an individual;
b. Measuring radio-labeled AM404 bound to receptors by positron emission tomography.

10. The method of claim 6, wherein said quantitating of radio-labeled endocannibinoids is by the additional step of quantitating radio-labeled endocannibinoid bound to receptors by positron emission tomography.

11. The method of claim 7, wherein said quantitating of radio-labeled endocannibinoids is by the additional steps:
a. Collecting sera from individuals;
b. Quantitating radio-labeled endocannibinoids by liquid chromatography.

12. The method of claim 7, wherein said radio-labeled cannibinoid includes ligands selected from the group consisting of: radio-labeled anandamide or 2-AG or their analogs that are functionally able to bind CB1 receptors.

13. The method of claim 7, wherein said detection of binding is by positron emission tomography.
